(19) United States
(12) Patent Application Publication
(10) Pub. No.: US 2022/0152060 A1
(43) Pub. Date: May 19, 2022

(54) PHYTOECDYSONES AND THE DERIVATIVES THEREOF FOR USE IN THE TREATMENT OF IMPAIRED LUNG FUNCTION

(71) Applicants: BIOPHYTIS, PARIS (FR); SORBONNE UNIVERSITE, PARIS (FR)

(72) Inventors: Mathilde LATIL, PARIS (FR); Pierre DILDA, PARIS (FR); René LAFONT, PARIS (FR); Stanislas VEILLET, SAVIGNY SUR ORGE (FR)

(21) Appl. No.: 17/439,669

(22) PCT Filed: Mar. 12, 2020
(86) PCT No.: PCT/EP2020/056591

§ 371 Date: Sep. 15, 2021

(30) Foreign Application Priority Data
Mar. 15, 2019 (FR) 1902727

(51) Int. Cl. A61K 31/58; A61P 11/00; A61K 31/575

(52) U.S. Cl. CPC A61K 31/58; A61K 31/575; A61P 11/00

(57) ABSTRACT

Disclosed are phytoecdysones and the derivatives thereof, intended for use in the treatment of impaired lung function in mammals, in particular in the context of a neuromuscular disease and more particularly when the impaired lung function is linked to a deterioration of the mechanical properties of the lung tissue.

### BACKGROUND OF THE INVENTION

### Field of the Invention

The invention relates to the use of phytoecdysones and semi-synthetic derivatives of phytoecdysones for the treatment of impaired respiratory function, in particular in the context of neuromuscular diseases.

### Description of the Related Art

Neuromuscular diseases are characterized by impaired function of the motor units, composed of motoneurons, neuromuscular junctions and skeletal muscles. In addition to the impaired motor function of patients with these pathologies, very many acute or progressive neuromuscular diseases lead to dysfunction of the respiratory muscles which, in turn, can lead to respiratory failure, pneumonia and death of the patients. Indeed, respiratory disorders are the main cause of death among patients with neurological diseases (Miller et al., 2009).

Patients with neuromuscular diseases may develop impaired respiratory function, which may be expressed, in an obvious manner, with the frequent occurrence of infections such as pneumonia or bronchitis (mainly due to the ineffectiveness of the cough), the feeling of shortness of breath, difficulty in expectorating. However, in some cases, the manifestations are less obvious and patients have a loss of appetite with severe weight loss, headaches, sweating or severe fatigue. Consequently, it is essential to detect respiratory disease as early as possible.

The care of respiratory problems in patients with neuromuscular pathologies has considerably improved in recent years, enabling increased life spans for patients, such as children with Duchenne muscular dystrophy. Respiratory parameters are regularly monitored using clinical examinations, spirometric imaging enabling exploration of respiratory function, or gasometry in order to evaluate the quality of gaseous exchange (measurement of the levels of oxygen and carbon dioxide gas in the arterial blood). These regular appraisals thus make it possible to detect the consequences of muscle weakness and pulmonary dysfunction and, thus, make it possible to adapt the medical care of these patients in order to compensate for their failing respiratory function and to improve their quality of life (Birnkrant et al., 2007; Finder et al., 2004; McKim et al., 2011). The care can be provided at several levels: it can be aimed at maintaining the mobility and flexibility of the respiratory apparatus (respiratory physiotherapy by active or passive mobilization or by mechanical hyperinsufflations) or at clearing the airways in order to remove secretions produced by the bronchi (assisted coughing or bronchial drainage) or, finally, when natural ventilation no longer meets the needs of the body, supplementing the breathing of the patients by non-invasive ventilation or, in the most extreme cases, through ventilation by tracheotomy.

Damage to the cortex, brain stem, spinal-cord, motoneurons, peripheral nerves, neuromuscular junctions or muscles can all lead to a failure of the respiratory system. There are many causes of chronic muscular diseases leading to a dysfunction of the respiratory muscles, including (congenital, hereditary or acquired) myopathies, myasthenia gravis or myotonia.

For example, in Duchenne muscular dystrophy (DMD), respiratory failure leading to numerous pulmonary complications is the cause of the majority of deaths observed in DMD patients (Mayer et al., 2015; Vianello et al., 1994; Baydur et al., 1990; Smith et al., 1987; Inkley et al., 1974). DMD is the most frequent form of muscular dystrophy. It affects one boy in 3500 and results from mutations affecting the dystrophin gene, located on the X chromosome. A less severe form, Becker muscular dystrophy (BMD), also involves the dystrophin gene and affects one boy in 18,000. Boys with DMD do not generally have difficulty breathing or coughing when they are still able to walk. As they get older and the disease affects their respiratory muscles, the affected boys risk contracting respiratory infections, often due to an ineffective cough. The smooth muscle cells and, by extension, the smooth muscles of the airways are implicated in numerous respiratory diseases.

In addition to the myopathic process, abnormalities of the pulmonary system itself (airways or lungs) are strongly involved in respiratory failure in DMD patients (Benditt and Boitano, 2013). Indeed, pulmonary compliance (capacity of the lung to modify its volume in response to a variation in pressure) is reduced for two main reasons: the retraction and collapse of the pulmonary alveoli (atelectasis) caused by hypoventilation, but also the appearance of fibrosis and obstruction of the airways, having as a consequence an increase in the resistance of the airways (Lo Mauro and Aliverti, 2016).

The elastic properties of the lung are impaired in patients with muscular dystrophy, the lung becoming less distensible. The cause of the reduction in pulmonary distensibility in muscular dystrophy is not yet fully understood. Nevertheless, various hypotheses have been proposed in order to attempt to explain the reduction in elasticity of the lungs: an incomplete development of the pulmonary tissue in the context of a congenital disease, hypoventilation-induced atelectasis, increase in the surface tension of the alveoli or damage to the pulmonary parenchyma due to fibrosis. Moreover, one of the significant factors for explaining this reduction in distensibility and pulmonary compliance is a low lung volume respiration, which is a characteristic of muscular dystrophy (Lo Mauro and Aliverti, 2016).

Another mechanism involved is the chronic and progressive degradation of the respiratory muscles which, de facto, limits the range of lung activity. Indeed, the elastic properties of a system are in part determined by the stresses to which the system is subjected. The total lung capacity is the result of the balance between the instantaneous elastic retraction pressure during ventilation and the pressure generated by the contraction of the inspiratory muscles. The latter is reduced and, consequently, the total lung capacity as well, which alters the expiration parameters and causes the reduction in pulmonary compliance.

To conclude, respiratory failure, characterized by the inability of the respiratory system to provide adequate oxygenation and removal of carbon dioxide, is common in patients with DMD.

Consequently, the evaluation of the failure of the respiratory system in mdx mice, the most used murine model of the Duchenne myopathy, is an important parameter to be considered in establishing and evaluating therapeutic solutions in the context of neuromuscular diseases.

Evaluating the respiratory system of mdx mice during preclinical studies has significant advantages: on the one hand, it involves a clinically important deficit and, on the other hand, the spirometric or plethysmographic measurements are non-invasive and can be repeated longitudinally during the trial and optionally performed as an evaluation criterion of the efficacy of various treatments for muscular dystrophy.

Various groups have been interested in the respiratory function of mdx mice in comparison with healthy control mice, and have reported impaired respiratory function in mdx mice (Gosselin et al. [2003]; Polizzi et al. [2003]; Polizzi et al. 2013; and Gayraud et al., [2007]). Hence, with some modifications to the severity of the impairments and the age at onset of these impairments, these all report a change in the respiratory parameters under normoxic conditions (Huang et al., 2011) or in the response to hypercapnia (Gosselin et al. 2003);

### SUMMARY OF THE INVENTION

The inventors have discovered that phytoecdysones and semi-synthetic derivatives of phytoecdysones significantly improve the respiratory function of mammals with neuromuscular disease, by limiting the change over time in the respiratory parameters, as well as by improving the mechanical parameters of the respiratory system. The respiratory parameters and the mechanical parameters of the respiratory system are determined respectively by whole-body plethysmography for awake animals and by piston ventilator controlled by a central control unit (commonly referred to as a “computer”) for anaesthetized animals, said ventilator using the forced oscillation technique as with the device known as FlexiVent™. These effects show an improvement in respiratory function in mammals with genetic or acquired, neuromuscular pathologies.

Phytoecdysones are an important family of polyhydroxylated phytosterols structurally related to insect molting hormones. These molecules are produced by many plant species and take part in their defense against insect pests. The major phytoecdysone is 20-hydroxyecdysone.

To this effect, the invention relates to at least one phytoecdysone and/or at least one semi-synthetic derivative of phytoecdysone, for use thereof in the treatment of impaired respiratory function.

The invention preferably relates to a composition comprising at least one phytoecdysone and/or at least one semi-synthetic derivative of phytoecdysone, for use thereof in the treatment of impaired respiratory function.

In particular embodiments, the invention also responds to the following characteristics implemented separately or in each of their technically feasible combinations.

The phytoecdysones and their derivatives are advantageously purified to pharmaceutical grade.

A phytoecdysone that is usable according to the invention is, for example, 20-hydroxyecdysone and a usable semi-synthetic derivative of phytoecdysone is, for example, a semi-synthetic derivative of 20-hydroxyecdysone.

To this effect, according to an embodiment, the composition includes 20-hydroxyecdysone and/or at least one semi-synthetic derivative of 20-hydroxyecdysone.

The 20-hydroxyecdysone and its derivatives are advantageously purified to pharmaceutical grade.

The 20-hydroxyecdysone used is preferably in the form of a plant extract rich in 20-hydroxyecdysone or a composition including 20-hydroxyecdysone as active ingredient. Plant extracts rich in 20-hydroxyecdysone are, for example extracts of Stemmacantha carthamoides (also called Leuzea carthamoides), Cyanotis arachnoidea and Cyanotis vaga.

The extracts obtained are preferably purified to pharmaceutical grade.

In an embodiment, the 20-hydroxyecdysone is in the form of plant extract or a plant part, said plant being chosen from plants containing at least 0.5% 20-hydroxyecdysone by dry weight of said plant, said extract including at least 95%, and preferably at least 97% 20-hydroxyecdysone. Said extract is preferably purified to pharmaceutical grade.

Hereinafter, said extract is referred to as 810101. It is characterized in that it includes between 0 and 0.05%, by dry weight of the extract, impurities, as minor compounds which may affect the safety, availability or efficacy of a pharmaceutical application of said extract.

According to an embodiment of the invention, the impurities are compounds with 19 or 21 carbon atoms, such as rubrosterone, dihydrorubrosterone or poststerone.

The plant from which 810101 is produced is preferably chosen from Stemmacantha carthamoides (also called Leuzea carthamoides), Cyanotis arachnoidea and Cyanotis vaga.

The phytoecdysone derivatives and in particular 20-hydroxyecdysone derivatives, are obtained by semi-synthesis and can be obtained, in particular, in the manner described in European patent application EP 15732785.9.

According to a preferred embodiment, the invention relates to the composition for use thereof in mammals in the treatment of impaired respiratory function, more particularly impaired respiratory function resulting from an acquired genetic neuromuscular pathology, for example a neuromuscular pathology of the motoneurons and/or of the neuromuscular junction and/or of the striated skeletal muscle.

According to a particular embodiment, the invention relates to the composition for the use thereof in mammals in the treatment of impaired respiratory function linked to impairment of the striated muscle and/or smooth muscle.

In a particular embodiment, the invention relates to the composition for the use thereof in mammals in the treatment of impaired respiratory function caused, at least in part, by modification of the smooth-muscle.

According to a particular embodiment, the invention relates to the composition for the use thereof in mammals in the treatment of impaired respiratory function linked to bronchial hyperreactivity.

In an embodiment, the invention relates to the composition for the use thereof in mammals in the treatment of impaired respiratory function wherein the bronchial hyperactivity is associated with the bronchial smooth muscle function.

In an embodiment, the invention relates to the composition for use thereof in mammals in the treatment of impaired respiratory function linked to a condition of at least one of the respiratory parameters chosen from the Penh value, peak inspiratory flow, peak expiratory flow, relaxation time, and respiratory rate. The composition will advantageously reduce the condition of these respiratory parameters.

In an embodiment, the invention relates to the composition for the use thereof in mammals in the treatment of impaired respiratory function linked to a condition of at least one of the mechanical parameters of the pulmonary tissue. Said mechanical parameters of the pulmonary tissue are the pulmonary elastance, compliance and resistance.

In a particular embodiment, the invention relates to the composition for the use thereof in mammals in the treatment of impaired respiratory function linked to a reduction in pulmonary compliance and/or an increase in pulmonary resistance and/or a reduction in pulmonary elastance.

In a particular embodiment, the invention relates to the composition for the use thereof in mammals in the treatment of a disease wherein impaired respiratory function is linked to retraction and collapse of the pulmonary alveoli and/or the onset of fibrosis.

In a particular embodiment, phytoecdysones are administered in a dose of between 3 and 15 milligrams per kilogram per day in humans. Here, phytoecdysone is understood to mean both phytoecdysones in general as well as their derivatives, 20-hydroxyecdysone (in particular in the form of an extract) and the derivatives thereof.

The phytoecdysones are preferably administered in a dose of 200 to 1000 mg/day, in one or more intakes, in an adult human, and a dose of 5 to 350 mg/day, in one or more intakes, in a human child or infant. Here, phytoecdysone is understood to mean both phytoecdysones in general as well as their derivatives, 20-hydroxyecdysone (in particular in the form of an extract) and the derivatives thereof.

In some embodiments, the composition includes at least one compound considered to be a derivative of phytoecdysone, said at least one compound being of general formula (I):

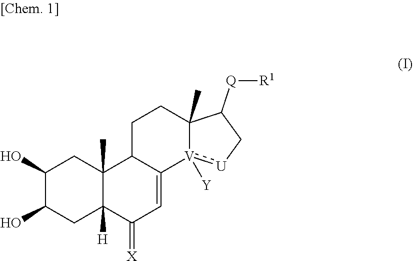

wherein:
V—U is a single carbon-carbon bond and Y is a hydroxyl group or a hydrogen atom, or V—U is an ethylene bond C═C;
X is an oxygen atom,
Q is a carbonyl group;
R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a hetero-ring optionally substituted by a group of type OH, MeO, (C₁-C₆), N(C₁-C₆), CO₂(C₁-C₆); a CH₂Br group;
W being a heteroatom chosen from N, O and S, preferably O and still more preferably S.

In the context of the present invention, “(C₁-C₆)” means any linear or branched alkyl group of 1 to 6 carbon atoms, in particular methyl, ethyl, n-propyl, iso-propyl, n-butyl, iso-butyl, sec-butyl, t-butyl, n-pentyl and n-hexyl groups. Advantageously it involves a methyl, ethyl, iso-propyl or t-butyl group, in particular a methyl or ethyl group, more particularly a methyl group.

In a preferred embodiment, in the formula (I):
Y is a hydroxyl group;
R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a hetero-ring optionally substituted by a group of type OH, MeO, (C₁-C₆), N(C₁-C₆), CO₂(C₁-C₆);
W being a heteroatom chosen from N, O and S, preferably O and more preferably S.

In some embodiments, the composition includes at least one compound chosen from the following compounds:
No. 1: (2S,3R,5R,10R,13R,14S,17S)-2,3,14-trihydroxy-10,13-dimethyl-17-(2-morpholinoacetyl)-2,3,4,5,9,11,12,15,16,17-decahydro-1H-cyclopenta[a]phenanthren-6-one,
No. 2: (2S,3R,5R,10R,13R,14S,17S)-2,3,14-trihydroxy-17-[2-(3-hydroxypyrrolidin-1-yl)acetyl]-10,13-dimethyl-2,3,4,5,9,11,12,15,16,17-decahydro-1H-cyclopenta[a]phenanthren-6-one;
No. 3: (2S,3R,5R,10R,13R,14S,17S)-2,3,14-trihydroxy-17-[2-(4-hydroxy-1-piperidyl)acetyl]-10,13-dimethyl-2,3,4,5,9,11,12,15,16,17-decahydro-1H-cyclopenta[a]phenanthren-6-one;
No. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
No. 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1H-cyclopenta[a]phenanthren-6-one;
No. 6: ethyl 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]sulfanylacetate;
No. 7: (2S,3R,5R,10R,13R,14S,17S)-17-(2-ethylsulfanylacetyl)-2,3,14-trihydroxy-10,13-dimethyl-2,3,4,5,9,11,12,15,16,17-decahydro-1H-cyclopenta[a]phenanthren-6-one;
No. 8: (2S,3R,5R,10R,13R,14S,17S)-2,3,14-trihydroxy-17-[2-[4-(2-hydroxyethyl sulfanyl)acetyl]-10,13-dimethyl-2,3,4,5,9,11,12,15,16,17-decahydro-1H-cyclopenta[a]phenanthren-6-one;

In some embodiments, the composition includes at least one compound considered to be a derivative of phytoecdysone, said at least one compound team of general formula (II):

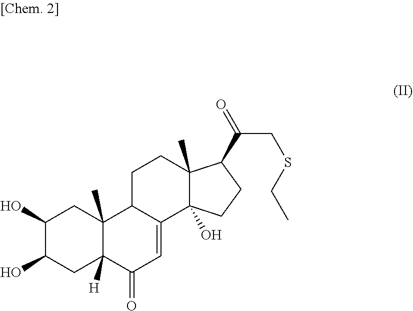

The compound of formula (II) is hereinafter referred to as BIO103.

In some embodiments, the composition is incorporated in a pharmaceutically acceptable formula that can be orally administered.

In the context of the present invention, “pharmaceutically acceptable” means that which can be used in the preparation of a pharmaceutical composition and which is generally safe, non-toxic and which is acceptable for veterinary as well as human pharmaceutical use.

### BRIEF DESCRIPTION OF THE DRAWINGS

The invention will be better understood on reading the following description, given by way of a non-limiting example, and with reference to the figures, wherein:

FIG. 1A shows the curve of the Penh value before the start of the study, for healthy control mice C57Black10 (n=12, white circles) and for mdx mice (n=23, white squares) before receiving the treatment (D0: day zero), measured by plethysmography, in response to increasing doses of metacholine with **p<0.001 and ***p<0.0001. In the rest of the description, n corresponds to the sample size and p corresponds to the “p-value” used to quantify the statistical significance of a result with *: p<0.05; **: p<0.001; ***p<0.0001 and ns: not significant.

FIG. 1B shows the curve of the peak inspiratory flow before the start of the study, for healthy control mice C57Black10 (n=12, white circles) and for mdx mice (n=23, white squares) before receiving the treatment (D0: day zero), measured by plethysmography, in response to increasing doses of metacholine with *p<0.05, **p<0.001;

FIG. 10 shows the curve of the peak expiratory flow before the start of the study, for healthy control mice C57Black10 (n=12, white circles) and for mdx mice (n=23, white squares) before receiving the treatment (D0: day zero), measured by plethysmography, in response to increasing doses of metacholine with **p<0.001;

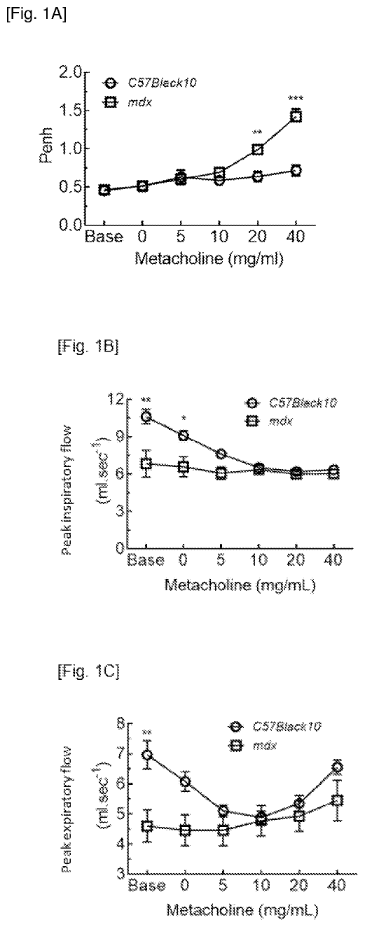

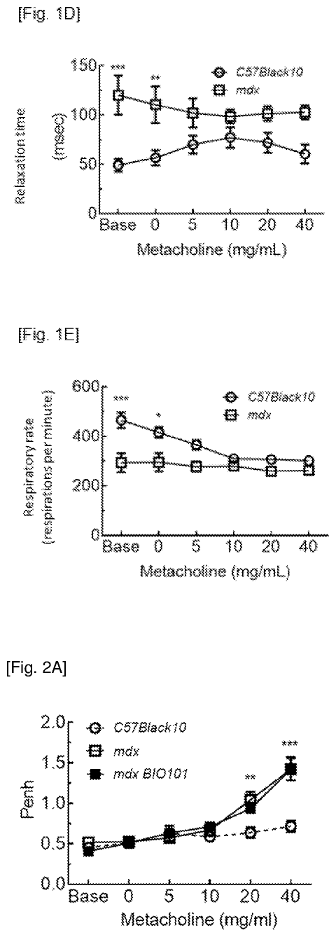

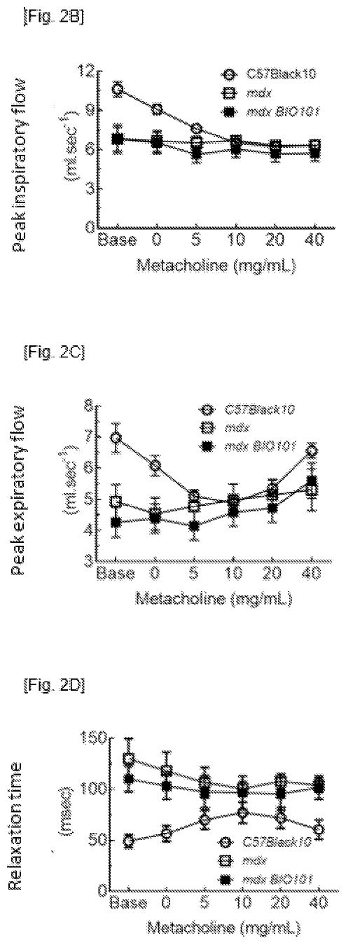

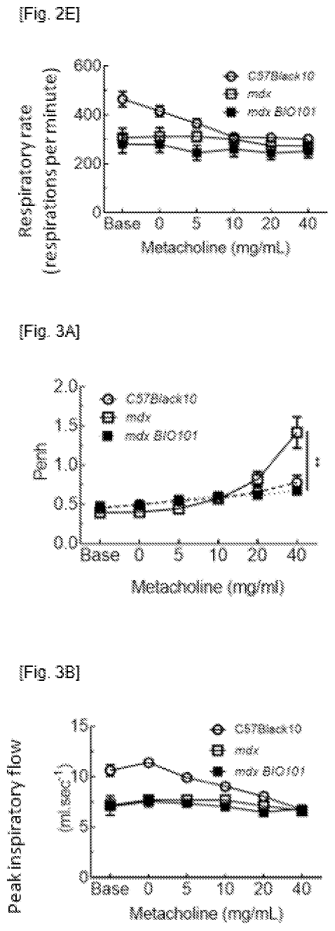

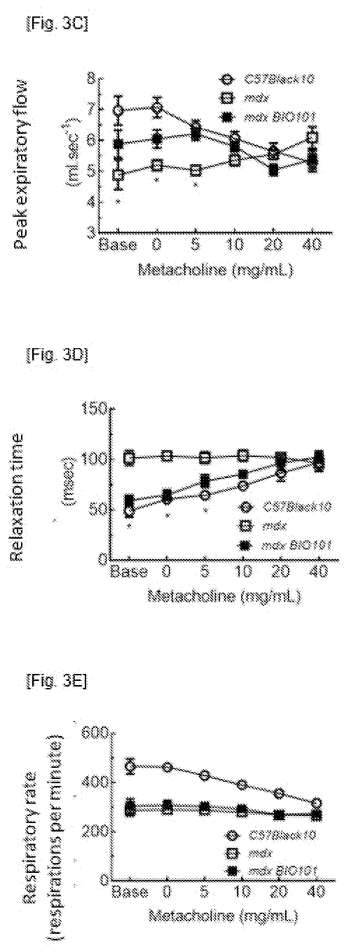

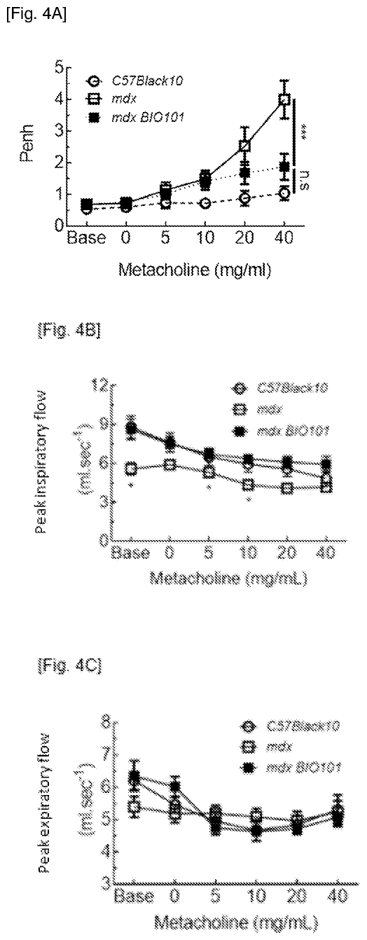

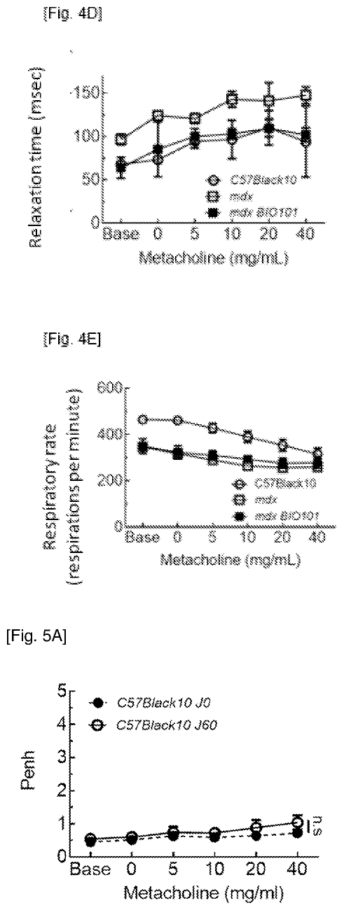

FIG. 1D shows the curve of the relaxation time before the start of the study, for healthy control mice C57Black10 (n=12, white circles) and for mdx mice (n=23, white squares) before receiving the treatment (D0: day zero), measured by plethysmography, in response to increasing doses of metacholine with **p<0.001 and ***p<0.0001.

FIG. 1E shows the curve of the respiratory rate before the start of the study, for healthy control mice C57Black10 (n=12, white circles) and for mdx mice (n=23, white squares) before receiving the treatment (D0: day zero), measured by plethysmography, in response to increasing doses of metacholine with *p<0.05 and ***p<0.0001.

FIG. 2A shows the curve of the Penh value before the start of the study (D0), measured by plethysmography, in response to increasing doses of metacholine, with **p<0.001 and ***p<0.0001, after the randomization of the animals in the three different groups: healthy control mice C57Black10 (n=12, white circles), mdx mice which will not receive treatment (n=11, white squares, “mdx” group) and mdx mice which will be treated with BIO101 (n=12, black squares, “mdx BIO101” group);

FIG. 2B shows the curve of the peak inspiratory flow before the start of the study (D0), measured by plethysmography, in response to increasing doses of metacholine, after randomization of the animals in the three different groups: healthy control mice C57Black10 (n=12, white circles), mdx mice which will not receive treatment (n=11, white squares, “mdx” group) and mdx mice which will be treated with BIO101 (n=12, black squares, “mdx BIO101” group);

FIG. 2C shows the curve of the peak expiratory flow before the start of the study (D0), measured by plethysmography, in response to increasing doses of metacholine, after randomization of the animals into the three different groups: healthy control mice C57Black10 (n=12, white circles), mdx mice which will not receive treatment (n=11, white squares, “mdx” group) and mdx mice which will be treated with BIO101 (n=12, black squares, “mdx BIO101” group);

FIG. 2D shows the curve of the relaxation time before the start of the study (D0), measured by plethysmography, in response to increasing doses of metacholine, after randomization of the animals in the three different groups: healthy control mice C57Black10 (n=12, white circles), mdx mice which will not receive treatment (n=11, white squares, “mdx” group) and mdx mice which will be treated with BIO101 (n=12, black squares, “mdx BIO101” group);

FIG. 2E shows the curve of the respiratory rate before the start of the study (D0), measured by plethysmography, in response to increasing doses of metacholine, after randomization of the animals in the three different groups: healthy control mice C57Black10 (n=12, white circles), mdx mice which will not receive treatment (n=11, white squares, “mdx” group) and mdx mice which will be treated with BIO101 (n=12, black squares, “mdx BIO101” group);

FIG. 3A shows the curve of the Penh value after 30 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with **p<0.001;

FIG. 3B shows the curve of the peak inspiratory flow after 30 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with *p<0.05, **p<0.001;

FIG. 3C shows the curve of the peak expiratory flow after 30 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with *p<0.05;

FIG. 3D shows the curve of the relaxation time after 30 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with *p<0.05;

FIG. 3E shows the curve of the respiratory rate after 30 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine;

FIG. 4A shows the curve of the Penh value after 60 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with ***p<0.0001;

FIG. 4B shows the curve of the peak inspiratory flow after 60 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine, with *p<0.05 cp;

FIG. 4C shows the curve of the peak expiratory flow after 60 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine;

FIG. 4D shows the curve of the relaxation time after 60 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine;

FIG. 4E shows the curve of the respiratory rate after 60 days of treatment, for healthy control mice C57Black10 (n=12, white circles), for untreated mdx mice (n=11, white squares, “mdx” group) and for mdx mice treated with BIO101 (n=12, black squares, “mdx BIO101” group), measured by plethysmography, in response to increasing doses of metacholine;

FIG. 5A shows the curve of the change in the Penh value measured by plethysmography, for healthy mice C57Black10 at the start of the study (n=12, black circles, D0) and at the end of the study (n=12, white circles, D60), subjected to increasing doses of metacholine;

FIG. 5B shows the curve of the change in the Penh value measured by plethysmography, for untreated mdx mice at the start of the study (n=23, black circles, D0) and at the end of the study (n=12, white circles, D60), subjected to increasing doses of metacholine, with ***p<0.0001;

FIG. 6A shows the Penh value at the start of the study (D0) for healthy mice C57Black10 (n=12) and for untreated mdx mice (n=23) measured by plethysmography with a metacholine dose of 40 mg/ml, with ***p<0.0001;

FIG. 6B shows the Penh values after 60 days of study (D60) for healthy mice C57Black10 (n=12), for untreated mdx mice (n=12) and for mdx mice treated with BIO101 (n=12) measured by plethysmography with a metacholine dose of 40 mg/mL, with ***p<0.0001;

FIG. 7A shows the change in pulmonary resistance measured by piston ventilator (FlexiVent™) at increasing doses of metacholine, in healthy mice C57Black10 (n=10), untreated mdx mice (n=10) and mdx mice treated with BIO101 (n=10), with **p<0.001 and ***p<0.0001;

FIG. 7B shows the change in pulmonary compliance measured by piston ventilator at increasing doses of metacholine, in healthy mice C57Black10 (n=10), untreated mdx mice (n=10) and mdx mice treated with BIO101 (n=10), with ***p<0.0001;

FIG. 7C shows the change in pulmonary elastance measured by piston ventilator at increasing doses of metacholine, in healthy mice C57Black10 (n=10), untreated mdx mice (n=10) and mdx mice treated with BIO101 (n=10), with ***p<0.0001;

FIG. 8A shows the values of pulmonary resistance after 60 days of study for healthy mice C57Black10 (n=10), for untreated mdx mice (n=10) and for mdx mice treated with BIO101 (n=10) measured by piston ventilator at a metacholine dose of 20 mg/mL, with ***p<0.0001;

FIG. 8B shows the values of pulmonary compliance after 60 days of study for healthy mice C57Black10 (n=10), for untreated mdx mice (n=10) and for mdx mice treated with BIO101 (n=10) measured by piston ventilator at a metacholine dose of 20 mg/mL, with ***p<0.0001;

FIG. 8C shows the values of pulmonary elastance after 60 days of study for healthy mice C57Black10 (n=10), for untreated mdx mice (n=10) and for mdx mice treated with BIO101 (n=10) measured by piston ventilator at a metacholine dose of 20 mg/mL, with **p<0.05 and **p<0.001;

### DESCRIPTION OF THE PREFERRED EMBODIMENTS

The invention will be described below in the particular context of some of its preferred non-limiting fields of application.

### 1. Method for Purifying BIO101

BIO101 is prepared from 90% pure 20-hydroxyecdysone, according to the following steps:
i) hot dissolving of 90% pure 20-hydroxyecdysone in methanol, filtering and the partial concentration,
ii) addition of 3 volumes of acetone,
iii) cooling to a temperature between 0 and 5° C., while stirring,
iv) filtering of the precipitate obtained,
v) successive rinsing with acetone and water, and
vi) drying.

This purification employs a recrystallization process appropriate for this molecule and capable of being performed on the industrial scale.

The filtration of step i) is performed using a 0.2 μm particle filter.

The partial concentration of step i) is advantageously carried out by vacuum distillation at a temperature of order 50° C. in the presence of MeOH.

Drying step vi) is carried out under vacuum at a temperature of order 50° C.

### 2. Biological Activity of BIO101

Male mice C57BL/10ScSnJ (healthy mice denoted as “C57Black10” in the figures) and C57BL/10ScSn-Dmdmdx/J (murine model of Duchenne muscular dystrophy, denoted “mdx” in the figures) aged 12 weeks, have been used. The mice have been divided into three groups of 12 mice each: an untreated group of healthy control mice C57Black10, a group of untreated mdx mice (mdx) and a group of mice chronically treated with a dose of 50 mg/kg/day, orally in the drinking water.

The respiratory function of all the mice has been evaluated by whole-body plethysmography before the start of the study, (D0) then at 30 days (D30) and 60 days (D60) after the start of the treatment. After two months of treatment, the respiratory function has been invasively evaluated, just before sacrifice of the animal, by piston ventilated controlled by a central control unit (commonly called “computer”), said ventilator using the forced oscillation technique, such as the device known by the name FlexiVent™, in order to determine more directly the mechanical parameters of the respiratory system.

a. Analysis by Plethysmography of the Effects of BIO101 on Respiratory Function

In order to precisely monitor the change in respiratory function over the course of the treatment, whole-body plethysmography analyses (Emka Technologies, Paris, France) have been performed on the healthy control mice C57Black10, on the untreated mdx mice and on the mdx mice having been treated with BIO101.

The advantages of this technique reside in the fact that it allows monitoring to be performed on an awake animal, freely moving in a hermetic enclosure, and that this is performed in a non-invasive manner. Consequently, the stress due to handling of the animals is reduced as it is possible to repeat the measurements over prolonged periods. Barometric plethysmography is therefore frequently used for measuring bronchial reactivity in small animals (Chong et al., 1998; Djuric et al., 1998; Hoffman et al., 1999).

The pressure variations measured with respect to a reference chamber make it possible to define various respiratory parameters such as the peaks and times of inspiratory and expiratory pressure as well as a dimensionless quantity called Penh (enhanced Pause) which allows the bronchoconstriction to be evaluated. More specifically, the Penh, calculated from the pressure signal (Pb) in the chamber, is an important index to obtain, because the variations in Penh change in parallel with those of the respiratory resistance and it therefore represents a predictive parameter for changes in the resistive properties of the respiratory system (Hamelmann et al., 1997; Bergren, 2001; Onclinx et al., 2003). The following values have been calculated from the filtered Pb: the maximum variation in Pb during expiration (PEP: Peak Expiratory Pressure), the maximum change in Pb during inspiration (PIP: Peak Inspiratory Pressure) and the time interval (TR). The Penh value has then been calculated as follows:

(PIP/PEP)×Pause [Math. 1]

where

Pause=(TE−TR)/TE [Math. 2]
TE being the expiration time (Adler et al., 2004).

Moreover, the peak inspiratory flow (PIF) and the peak expiratory flow (PEF), the relaxation time (RT) and the respiratory rate (BF) have also been measured and shown.

The Penh value has been measured before the start of the treatment (D0), 30 days after the start of the treatment (D30) and 60 days after the treatment (D60).

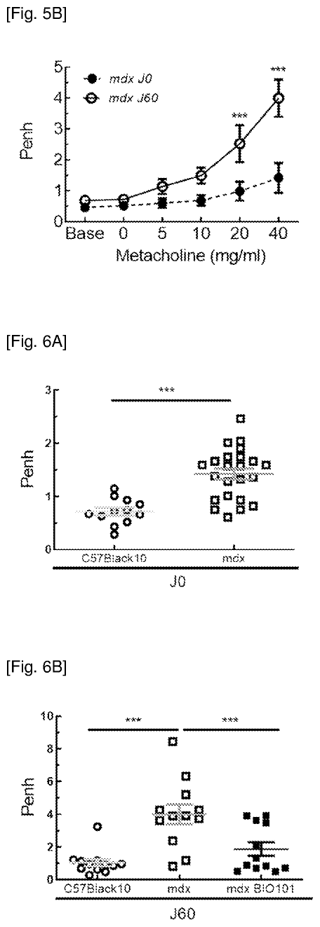

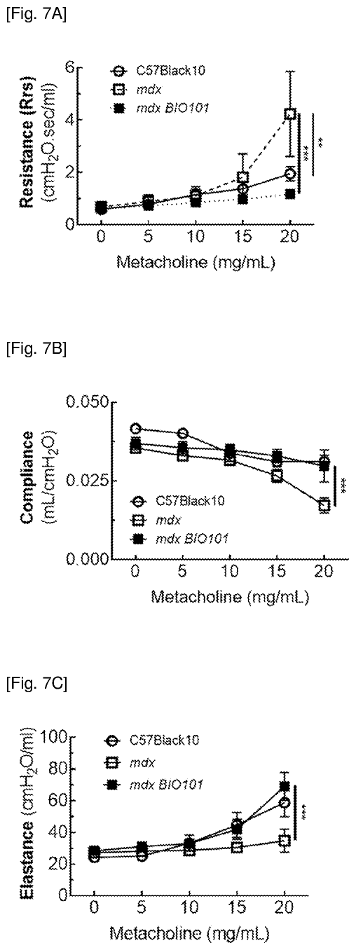

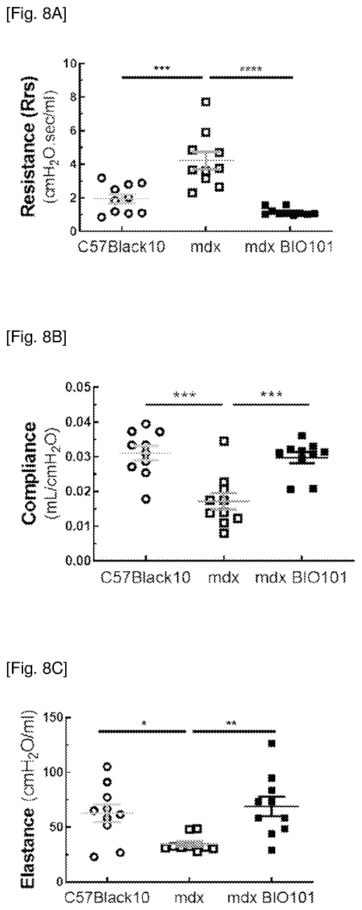

First, the entire cohort of mdx mice (n=24) were compared with healthy mice C57Black10 (n=12), before receiving the treatment. Awake mice were exposed to increasing doses of metacholine aerosols generated by a nebulizer containing 0 to 40 mg/mL of metacholine in PBS. As expected and already demonstrated, the mdx mice exhibit impaired respiratory function (Huang et al., 2011; Gosselin et al., 2003; Gayraud et al., 2007; Ishizaki et al., 2008) with a significant increase in the Penh in response to metacholine (p<0.001 and p<0.0001) (FIG. 1A) associated with a significantly lower peak inspiratory flow and peak expiratory flow (p<0.05 and p<0.001) (FIGS. 1B and 1C) in comparison with healthy mice, in particular before or at low bronchoconstrictor dose. The relaxation time (RT) is also very significantly greater in mdx mice in comparison with the control mice (p<0.001 and p<0.0001) (FIG. 10). The values of inspiration time (TI) and expiration time (TE) remain comparable between the groups at each measurement time (data not shown). The respiration rate of the mdx mice is also reduced compared with the healthy mice (p<0.05 and p<0.0001) (FIG. 1E).

The mdx mice were then divided into two different groups: the mdx mice which were not going to receive any treatment (white squares) and the mdx mice which were going to receive the molecule BIO101 (black squares). As shown in FIGS. 2A, 2B, 2C, 2D and 2E, these two groups exhibit the same capacities for their respiratory function without significant difference between the various respiratory parameters measured, which shows that before treatment, all the untreated mdx mice have the same respiratory profile.

After 30 days of treatment, the mdx mice treated with BIO101 exhibit a significant reduction in Penh (p<0.001) compared to the mdx mice at D0, with a curve of the Penh in response to metacholine comparable to that of healthy mice C57Black10 (FIG. 3A). The reduction in Penh is associated with an increase in peak inspiratory flow, in particular in the equilibrium state or at low metacholine dose (p<0.05) (FIG. 3C). Moreover, a clear improvement in relaxation time is observed in the mdx mice treated with BIO101 compared with the untreated mdx mice (p<0.05) (FIG. 3D). By contrast, the peak inspiratory flow and the respiratory rate remain unchanged in comparison to that measured at D0 (FIGS. 3B and 3E).

After 60 days of treatment, BIO101 maintains its beneficial effect on the respiratory function of the mdx mice. Specifically, the results confirm that which is observed after 30 days of treatment. The mdx mice treated with BIO101 show significant reduction in the Penh compared to the untreated mdx mice (p<0.0001) and the profile of the variations in Penh is similar to that of the control, whatever the metacholine dose (FIG. 4A). A significant improvement is seen in the peak inspiratory flow (p<0.05) in conjunction with a trend towards improved relaxation time (FIGS. 4B and 4D). By contrast, the peak expiratory flow and the respiratory rate remain unchanged (FIGS. 4B and 4E).

In order to longitudinally evaluate impaired respiratory function in mdx mice, the variation in Penh was compared between D0 and D60 in the healthy control mice as well as between D0 and D60 in untreated mdx mice. As expected, the Penh has not changed between D0 and D60 in the control mice (p=ns) (FIG. 5A). By contrast, respiratory function is significantly degraded in the mdx mice at D60 compared with D0, as shown by the increase in Penh in response to metacholine (p<0.0001) (FIG. 5B).

At a metacholine dose of 40 mg/mL, the mean value of the Penh in healthy control mice (n=12) is significantly less than that found in mdx mice (n=23) before treatment (Penh=0.72 and 1.42 respectively, with p<0.0001) (FIG. 6A). At D60, this Penh value is higher in the untreated mdx mice (n=12) compared to the healthy control mice (n=12) with values of 4 and 1.04 respectively (p<0.0001) (FIG. 6B). Interestingly, the treatment with BIO101 significantly reduces this value, with a Penh value equal to 1.87 (p<0.0001), compared to the untreated mdx mice (FIG. 6B). The treatment reduces the Penh value of the mdx mice to a level not significantly different from that of healthy mice (Penh=1.04 in C57Black10 mice versus 1.87 in mdx mice, with p<0.05).

b. Analysis by Piston Ventilator of the Effects of BIO101 on Pulmonary Resistance, Compliance and Elastance

In order to determine more directly the mechanical parameters of the respiratory system and the impact of a chronic treatment over a period of two months by BIO101 (D60) on these parameters, the dynamic pulmonary resistance was measured using a piston ventilator system as previously described, in response to increasing doses of metacholine.

The mice were anaesthetized and connected by an endotracheal cannula to the piston ventilator system. After the start of mechanical ventilation, each mouse received an intraperitoneal injection of 0.1 mL of a 10 mg/mL rocuronium bromide solution. The animal was ventilated at a respiratory rate of 150 respirations/minute and with a tidal volume of 10 mL/kg against a final positive expiratory pressure of 3 cm H₂O. The respiratory mechanisms were evaluated using a forced oscillation maneuver with a duration of 1.2 seconds (2.5 Hz) and a 3-second wideband forced oscillation man oeuvre containing 13 primordial frequencies between 1 and 20.5 Hz. The resistance of the respiratory system (R) was calculated using a microcomputer type central processing unit connected to the piston ventilator system and comprising, in particular, software for performing said calculation. The two maneuvers were carried out alternately every 15 seconds after each nebulization with a metacholine aerosol, in order to measure the change in the response time of the bronchoconstriction induced by metacholine. The pulmonary resistance, compliance and elastance could thus be determined.

In line with the results obtained by plethysmography, it was observed that the untreated mdx mice have a resistance of the airways greater than that of healthy mice C57Black10 (p<0.001) (FIG. 7A). After 60 days of treatment, in response to increasing doses of metacholine from 0 to 20 mg/mL, the mdx mice treated with BIO101 exhibit a significantly lower resistance of the airways than the untreated mdx mice (p<0.0001), comparable to the level of pulmonary resistance observed in the healthy control mice (FIG. 8A). In the same way, the chronic treatment over two months with BIO101 significantly improved the pulmonary compliance (p<0.0001) and elastance (p<0.0001), two parameters which were altered in the mdx mice (FIGS. 7B and 7C). Specifically, at the highest metacholine dose tested (20 mg/mL), the mdx mice exhibit a significantly reduced pulmonary compliance compared to the control mice C57Black10 (0.017 mL/cmH₂O and 0.031 mL/cmH₂O respectively, with p<0.0001). This pulmonary compliance defect is significantly corrected with the treatment by BIO101 for the mdx mice (0.029 mL/cmH₂O, with p<0.0001) (FIG. 8B). In an identical manner, the pulmonary elastance is greatly reduced in the mdx mice compared to the control mice C57Black10 (34.8 cmH₂O/mL versus 62.8 cmH₂O/mL, with p<0.05) and the treatment by BIO101 maintains the pulmonary elastance similar to that for healthy control mice and significantly greater than the untreated mdx mice (34.8 cmH₂O/mL versus 69.1 cmH₂O/mL, with p<0.001) (FIG. 8C).

### 3. Conclusion

These results show that the treatment by BIO101 (50 mg/kg per day in the drinking water) improves the respiratory function of mdx mice (an animal model for Duchenne muscular dystrophy) and does so in a prolonged manner over time. This effect on the respiratory function is not only associated with the respiratory parameters (inspiratory and expiratory duration and frequency), as demonstrated by the plethysmography results and, in particular, the measurements of the Penh, but also demonstrate an improvement in the structure of the deep airways, demonstrated by the experiments using the piston ventilator system. Indeed, the data of this system significantly demonstrate beneficial effects of the treatment by BIO101 on the mechanical respiratory parameters of resistance, compliance and elastance of the lungs in mdx mice. These observations are a consequence of a protection through the treatment with phytoecdysones, in particular BIO101, from degradation of the lung functions over time, in a murine model of neuromuscular pathology.

More generally, it should be noted that the embodiments of the invention considered above have been described by way of non-limiting examples, and that other variants can consequently be envisaged.

1. A method of treatment of impaired respiratory function resulting from an acquired or genetic neuromuscular disease, or impaired respiratory function linked to bronchial hyperreactivity in mammals, said method comprising the step of administering a therapeutic dose of a composition comprising at least one phytoecdysone and/or at least one semi-synthetic derivative of phytoecdysone to a subject in need thereof.

2. The method according to claim 1, wherein the composition includes 20-hydroxyecdysone and/or at least one semi-synthetic derivative of 20-hydroxyecdysone.

3. The method according to claim 2, wherein the 20-hydroxyecdysone is in the form of plant extract or a plant part, said plant being chosen from plants containing at least 0.5% 20-hydroxyecdysone by dry weight of said plant, said extract including at least 95% 20-hydroxyecdysone.

4. The method according to claim 3, wherein the composition comprises between 0 and 0.05%, by dry weight of the extract, impurities which may affect the safety, availability or efficacy of a pharmaceutical application of said extract.

5. The method according to claim 3, wherein the plant is chosen from Stemmacantha carthamoides, Cyanotis arachnoidea and Cyanotis vaga.

6. The method according to claim 1, wherein the impaired respiratory function results from neuromuscular disease of the motoneurons and/or of the neuromuscular junction and/or of the striated skeletal muscle.

7. The method according to claim 1, wherein the impaired respiratory function is linked to an impairment of the striated muscle and/or smooth muscle.

8. The method according to claim 1, wherein the bronchial hyperreactivity is associated with bronchial smooth muscle function.

9. The method according to claim 1, wherein the impaired respiratory function is linked to a condition of at least one of the respiratory parameters chosen from the Penh value, peak inspiratory flow, peak expiratory flow, relaxation time, and respiratory rate.

10. The method according to claim 1, wherein the impaired respiratory function is linked to a condition of at least one of the mechanical parameters of the lung tissue.

11. The method according to claim 10, wherein the impaired respiratory function is linked to a reduction in pulmonary compliance and/or an increase in pulmonary resistance and/or a reduction in pulmonary elastance.

12. The method according to claim 1, wherein the phytoecdysones are administered in a dose between 3 and 15 milligrams per kilogram per day in humans.

13. The method according to claim 1, wherein the phytoecdysones are administered in a dose of 200 to 1000 mg/day, in one or more intakes, in an adult human, and a dose of 5 to 350 mg/day, in one or more intakes, in a human child or infant.

14. The method according to claim 1, wherein the composition comprises at least one compound of general formula (I): 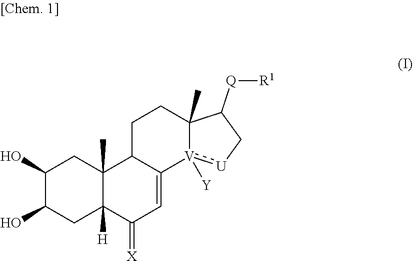
wherein:
V—U is a single carbon-carbon bond and Y is a hydroxyl group or a hydrogen atom, or V—U is an ethylene bond C═C;
X is an oxygen atom,
Q is a carbonyl group;
R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a hetero-ring; and a CH₂Br group;
W being a heteroatom chosen from N, O and S.

15. The method according to claim 14, wherein in the general formula (I):
Y is a hydroxyl group;
R¹ is chosen from:
a (C₁-C₆)W(C₁-C₆) group;
a (C₁-C₆)W(C₁-C₆)W(C₁-C₆) group;
a (C₁-C₆)W(C₁-C₆)CO₂(C₁-C₆) group; and
a (C₁-C₆)A group, A representing a hetero-ring;
W being a heteroatom chosen from N, O and S.

16. The method according to claim 14, wherein said at least one compound of general formula (I) is chosen from:
No. 1: (2S,3R,5R,10R,13R,14S,17S)-2,3,14-trihydroxy-10,13-dimethyl-17-(2-morpholinoacetyl)-2,3,4,5,9,11,12,15,16,17-decahydro-1H-cyclopenta[a]phenanthren-6-one;
No. 2: (2S,3R,5R,10R,13R,14S,17S)-2,3,14-trihydroxy-17-[2-(3-hydroxypyrrolidin-1-yl)acetyl]-10,13-dimethyl-2,3,4,5,9,11,12,15,16,17-decahydro-1H-cyclopenta[a]phenanthren-6-one;
No. 3: (2S,3R,5R,10R,13R,14S,17S)-2,3,14-trihydroxy-17-[2-(4-hydroxy-1-piperidyl)acetyl]-10,13-dimethyl-2,3,4,5,9,11,12,15,16,17-decahydro-1H-cyclopenta[a]phenanthren-6-one;
No. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
No. 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1H-cyclopenta[a]phenanthren-6-one;
No. 6: ethyl 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]sulfanylacetate;
No. 7: (2S,3R,5R,10R,13R,14S,17S)-17-(2-ethylsulfanylacetyl)-2,3,14-trihydroxy-10,13-dimethyl-2,3,4,5,9,11,12,15,16,17-decahydro-1H-cyclopenta[a]phenanthren-6-one;
No. 8: (2S,3R,5R,10R,13R,14S,17S)-2,3,14-trihydroxy-17-[2-(2-hydroxyethylsulfanyl)acetyl]-10,13-dimethyl-2,3,4,5,9,11,12,15,16,17-decahydro-1H-cyclopenta[a]phenanthren-6-one.

17. The method according to claim 1, wherein the composition comprises at least one compound of general formula (II):

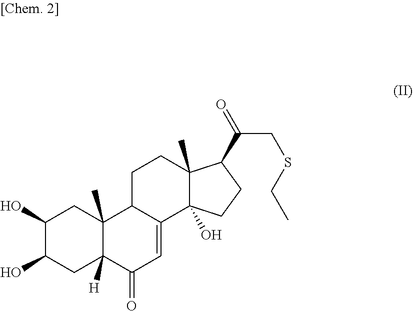

18. The method according to claim 2, wherein the 20-hydroxyecdysone is in the form of plant extract or a plant part, said plant being chosen from plants containing at least 0.5% 20-hydroxyecdysone by dry weight of said plant, said extract including at least 97%, 20-hydroxyecdysone.

19. The method of claim 14, wherein W is O.

20. The method of claim 14, wherein W is S.
